(19) United States
(12) Patent Application Publication
(10) Pub. No.: US 2012/0077684 A1
(43) Pub. Date: Mar. 29, 2012

(54) Methods and Compositions Including Diagnostic Kits For The Detection In Samples Of Methicillin-Resistant Staphylococcus Aureus

(76) Inventor: Shawn Mark O'Hara, Richboro, PA (US)

(21) Appl. No.: 12/809,090

(22) PCT Filed: Dec. 26, 2008
(86) PCT No.: PCT/US08/88356

§ 371 Date: Dec. 18, 2011

Related U.S. Application Data
(60) Provisional application No. 61/009,125, filed on Dec. 26, 2007.

(51) Int. Cl. C40B 30/00; C12Q 1/68

(52) U.S. Cl. 506/7; 435/6.12

(57) ABSTRACT

The present invention provides methods, compositions and diagnostic kits for the detection of Staphylococcus Aureus (SA) and antibiotic resistant forms and variants thereof, such as methicillin-resistant Staphylococcus aureus (MRSA), vancomycin-resistant Staphylococcus aureus (VRSA), mupirocin-resistant Staphylococcus aureus (mupSA), and the like, in a sample population. The invention preferably involves the improvements of bacterial sampling by means of SA enrichment, followed by SA cell disruption and amplification procedures incorporating the use of multiplex assays for SA specific genes, such as mecA and coagulase negative Staphylococci (CONS) specific genes such as tufA, for SA identification and identification of its known species. This provides means for controlling for the thirty or more known CONS species in assessing SA samples, especially those CONS species that may carry antibiotic resistance genes, such as SCCmec.

### CROSS REFERENCE TO RELATED APPLICATIONS

This application is a non-provisional application, which is incorporated by reference herein and claims priority, in part, of U.S. Provisional Application No. 61/009,125), filed 26 Dec. 2007.

### BACKGROUND OF THE INVENTION

1. Field

The present invention relates to novel methods, compositions and antibiotic resistant forms and variants thereof, such as methicillin-resistant Staphylococcus aureus (MRSA), vancomycin-resistant Staphylococcus aureus (VRSA), mupirocin-resistant Staphylococcus aureus (mupSA), and the like, in a sample population.

2. Background Art

Staphylococcus Aureus (SA) is a major cause of skin, soft tissue, and bloodstream infections that may become rapidly fatal to infected individuals if not treated effectively. SA and methicillin-resistant Staphylococcus aureus (MRSA) are now endemic in many hospitals in the United States and other countries. The incidence of disease across the United States from antibiotic-resistant forms of SA is expected to continue to increase. Recently, a study by the Centers for Disease Control and Prevention, (CDC) demonstrated that by 2005 there were more deaths related to invasive MRSA disease than from HIV-AIDS. Colonization (defined as nasal carriage only) with SA, MRSA, VRSA, and the like is associated with eventual infection. These infections have high medical care cost and generally result in poor clinical outcomes. With an increased burden of in-hospital MRSA-related disease and the emerging concern that community-associated (CA) MRSA will continue to increase, medical professionals and the public are urgently seeking a rapid and cost effective means to limit the spread of these pathogens. The CDC study indicated that 85% of invasive MRSA infections are still healthcare treatment-associated, suggesting that better hospital programs to address this problem would be necessary for helping to stop this pathogen from becoming more widely spread.

SA has become the single leading pathogen in health care-associated infections. Nasal carriage of SA has been postulated as a source of bacteremia, surgical-site, and other infections and as a reservoir of SA in hospitals. Early detection of nasal carriage (colonization) and cost effective diagnosis has been shown to prevent the spread of infections, reduce transmission and reduce net hospital costs.

Screening patients for SA colonization using culture methods is time consuming and generally requires 1 to 4 or more days for accurate detection and identification of SA. However, it is possible to obtain results within two hours using real-time polymerase chain reaction (PCR) assays in detecting SA (see, for example, “Direct Detection of Staphylococcus aureus from Adult and Neonate Nasal Swab Specimens Using Real-Time Polymerase Chain Reaction,” Paule, S. M., Pasquariello A. C., Hacek, D. M. Fisher A. G., Thomson, R. B., Kaul, K. L., and Peterson, L. R., J. Molecular Diagnostics, Vol 6, No. 3; pgs. 191-196, 2004 and “New Real-Time PCR Assay for Rapid Detection of Methicillin-Resistant Staphylococcus aureus Directly from Specimens Containing a Mixture of Staphylococci,” A. Huletsky, R. Giroux, V. Rossbach, M. Gagnon, M. Vaillancourt, M. Bernier, F. Gagnon, K. Truchon, M. Bastien, F. J. Picard, A. van Belkum, M. Ouellette, P. H. Roy, and M. G. Bergeron, J. CLIN MICRO, Vol. 42, No. 5; pgs. 1875-1884, May 2004).

Consequently, there is a need for a cost-effective method for the rapid detection of SA as a diagnostic tool in the detection, prevention, and treatment of this contagious disease. PCR assays to detect nasal colonization of SA have the potential to obtain information in less than 1 hour. A rapid PCR assay as a first step in a population sampling strategy to screen patients for SA would enable significant cost savings, especially when screening for the antibiotic resistant forms of SA such as MRSA, VRSA and the like.

Methicillin resistance in SA is caused by the acquisition of an exogenous gene, mecA, that encodes an additional B-lactam-resistant penicillin-binding protein (PBP), termed PBP 2a (or PBP2′). The mecA gene is carried by a mobile genetic element, designated staphylococcal cassette chromosome mec (SCCmec), inserted near the chromosomal origin of replication. The SCCmec DNAs are integrated at a specific site (attBscc) adjacent to orfX gene in the methicillin-susceptible S. aureus (MSSA) chromosome.

Techniques for detecting MRSA using nasal swabs and real-time PCR testing have increased the speed and accuracy for identification of SA and confirmation of its antibiotic resistant forms such as MRSA, VRSA and the like. Multiplex PCR incorporating the detection of the mecA and femA genes has been used in diagnosis of MRSA from colonies isolated from nasal cultures. Similarly, this multiplex approach has been used successfully for identifying MRSA directly from mixed staphylococcus nasal swab samples following immunomagnetic enrichment of SA from these nasal samples (see, for example, “Rapid Detection of Methicillin-Resistant Staphylococcus aureus Directly from Sterile or Nonsterile Clinical Samples by a New Molecular Assay,” Patrice Francois, Didier Pittet, Manuela Bento, Be'atrice Pepey, Pierre Vaudaux, Daniel Lew and Jacques Schrenzel, J. CLIN MICRO, Vol. 41, No. 1; pgs. 254-260, January 2003). More recently, PCR techniques for identifying the SA SCCmec insertion site have enabled the detection of MRSA directly from mixed Staphylococcal nasal samples without the need for SA enrichment or colony isolation. However, it is also important to note that the SCCmec approach has approximately an inherent 5% false positive rate. Recently the United States FDA approved 2 versions of the SCCmec PCR assay, as shown in Table 1 infra. However, broad adoption by healthcare providers and active surveillance using these 2 SCCmec based assays has generally been cost prohibitive. The high overall cost of MRSA screening using these 2 SCCmec assays is due in large part to their elaborate sample preparation methods and lack of test population stratification, as 70-75% can be ruled out with a much less expensive and rapid test for SA-positive sample stratification prior to a subsequent rapid MRSA verification test. Thus, in spite of the availability of accurate MRSA PCR assays, there still exists a need to provide cost-effective and rapid detection of SA for use in the diagnosis of its antibiotic-resistant forms.

In U.S. patent application Ser. No. 10/471,819 there is described a procedure for the detection and identification of MRSA directly from a sample such as nose or inguinal swabs. After rapid conditioning of the sample, a two step process of enrichment and amplification involving mecA and femA genes is employed in a selection process incorporating methicillin resistance (mecA) followed by femA genes for S. Aureus and S. epidermidis. See also, for example, the previously cited Francois, et al article wherein a triplex qPCR assay after immunomagnetic enrichment assay is described. These disclosures describe simultaneous target detection of the mecA gene conferring methicillin resistance, common to both S. aureus and Staphylococcus epidermidis, femA gene from S. aureus and fern A gene from S. epidermidis.

In addition, previous techniques known in the art have focused on the use of topical nasal antimicrobial agents to decolonize samples taken from patients, which have been known to result in the PCR assay detecting nonviable MRSA. The use of antibiotics interferes with enrichment, such as protein A enrichment, and is known to shift the balance of MRSA present in samples to methicillin resistant coagulase negative staphylococci species, leaving any or all of the other non-SE species as the dominant population. In contrast, the present invention addresses in part this problem, by identifying and controlling for all presently known non-SE methicillin-resistant, coagulase negative staphylococci species.

### SUMMARY OF THE INVENTION

The present invention provides methods, compositions and diagnostic kits for the detection of Staphylococcus Aureus (SA) and antibiotic resistant forms and variants thereof, such as methicillin-resistant Staphylococcus aureus (MRSA), vancomycin-resistant Staphylococcus aureus (VRSA), mupirocin-resistant Staphylococcus aureus (mupSA), and the like, in a sample population. The invention preferably involves the improvements of bacterial sampling by means of SA enrichment, followed by SA cell disruption and amplification procedures incorporating the use of multiplex assays for SA specific genes, such as mecA and coagulase negative Staphylococci (CONS) specific genes such as tufA, for SA identification and identification of its known species. This provides means for controlling for the thirty or more known CONS species in assessing SA samples, especially those CONS species that may carry antibiotic resistance genes, such as SCCmec.

Accordingly, the present invention provides novel methods, compositions and diagnostic kits which can enable cost effective management and control for the detection and diagnosis of SA and any of its antibiotic-resistant forms and variants thereof. In a preferred embodiment, the present invention also provides improved methods and kits for detection of Methicillin-Resistant Staphylococcus aureus (MRSA). The present invention utilizes the mecA gene and the femA gene from SA, and in a further preferred embodiment contemplates the use of nuc137 therefrom. Further, the present invention contemplates incorporation of the tufA target gene in place of the femA from S. epidermidis (SE), which enables the further identification of the presence of any and/or all of the presently known species of coagulase negative Staphylococci (CONS), rather than the identification of the single CON species of SE.

Accordingly, an objective of the improved methods, compositions and diagnostic kits of the present invention is to address the surprisingly high false positive rates reported as described in the above referenced art.

A further objective of the present invention is to provide improved methods, compositions and diagnostic kits for controlling and identifying the presence of all species of coagulase negative methicillin-resistant Staphylococci (MRCONS), which is provided by utilization of the tufA consensus gene sequence.

A still further objective of the present invention is to provide for the use of different SA antibody-antigen complexes forming combinations, to selectively enrich for SA and to selectively eliminate all of the known species, and as yet any unknown, MRCONS species.

Additional objectives and advantages of the present invention will be apparent to those skilled in the art, considering the descriptions of preferred embodiments of the invention set forth herein.

### THE DRAWINGS

FIG. 1 is a flow chart depicting a preferred, general nasal swab MRSA carriage assay procedure in accordance with the present invention, using enrichment options with a MRCONS consensus assay for tufA, in combination with mecA and femA-SA (or nuc), and MRSA PCR detection using a DNA derived from a mucosal sample without isolation of the sample DNA from disrupted SA cells.

FIG. 2 shows a GenBank orfX gene map, showing the relevant gene linkage in MRSA strain USA300. This map illustrates the binding pair (bp) distance of about 7412 bp between the extremes of the genes orfX 33721 bp and mecA 41133 bp, equal to 7412 bp maximum separation distance. Thus, the presence of SA and or SE can be identified by species specific orfX PCR. The presence of MRSA and MRSE can be identified in routine gDNA fragment pools averaging 20-23 kb by a combination of hybrid probe capture and enrichment followed by multiplex quantitative PCR determining the relative ratios of three markers: SA-orfX, SE-orfX and SCCmecA.

### DETAILED DESCRIPTION OF THE PREFERRED EMBODIMENTS

Staphylococcus aureus (SA) and methicillin-resistant Staphylococcus aureus (MRSA) is now endemic in many United States hospitals. The burden of SA infections on hospitals in the United States has recently been demonstrated in recent reports showing that SA infections were reported in patient discharge diagnosis for 0.8% of all hospital inpatients, or 292,045 stays per year. Inpatients with SA infection had, on average, three times the length of hospital stay compared to inpatients without this infection (14.3 vs 4.5 days; P=0.001), 3 times the total charges ($48 vs $14; P=0.001), and 5 times the risk of in-hospital death (11.2% vs 2.3%; P=0.001). Even when controlling for hospital-fixed effects and for patient differences in diagnosis-related groups, age, sex, race, and co-morbidities, the differences in mean length of stay, total charges, and mortality were significantly higher for hospitalizations associated with SA. The potential benefits to hospitals in terms of reduced use of resources and costs as well as improved outcomes from preventing SA, MRSA and VRSA infections are significant. The high costs of current tests for determining these pathogens may be mitigated by procedures that would rule out about 70% of the samples by using a much less expensive test. Accordingly, it has been found that in accordance with the present invention, methods, compositions and diagnostic kits have been developed that effectively streamline sample preparation, and utilize SA prevalence, in order to provide more cost effective alternatives to the techniques of the conventional art. Table 1, below, shows a comparison of example commercial assays in accordance with the conventional art:

TABLE 1
Comparison of Commercial MRSA Assays

| | | |
|---|---|---|
| Feature | Cepheid GeneXpert $45 ea. | BD |
| | | GeneOhm* $30 ea |
| Instrumentation | Cepheid GeneXpert | PCR |
| | Dx System | equipment |
| Fluidics | Self-contained and | Multiple |
| | automated after swab elution | manual steps |
| | and 2 single-dose reagents | |
| Lysis | Sonication (automated | Glass beads |
| | single-use cartridge) | (manual) |
| DNA Target | Sequences incorporating the | Sequence |
| | Sequence insertion site | near the insertion |
| | (AttBssc) of SCCmec | site of SCCmec |
| Internal Controls | Sample processing control | Internal |
| | and probe check control | control |
| Time to Result | 75 minutes | 60 to 75 |
| | | minutes |
| Users | Operators with little clinical | CLIA high |
| | lab experience to experienced | complexity |
| | lab technologists | technologists |

*Originally marketed as the IDI-MRSA.
Source: FDA 510(k) summary

In contrast to conventional assays, the present invention utilizes a sampling algorithm and Direct PCR from SA disrupted nasal swabs as samples in a commercially available, FDA approved PCR kit such as the above described. Direct nasal SA DNA sample preparation without DNA isolation for PCR provides a faster and less expensive screening method for SA in health care settings. In a preferred embodiment, the present invention also focuses on population prevalence of SA relative to MRSA, VRSA, ORSA, or CONS/CoNS. For example, SA has been determined to be well established and prevalent in the general population at around 30%, compared to MRSA which is present at approximately 0.8%. In hospitals, SA prevalence remains at approximately 30% while the proportion of MRSA can increase dramatically within its SA population, potentially rising to 60% of the SA population. The present invention thus provides an improved strategy for MRSA screening, utilizing direct PCR for the much simpler and cheaper SA analysis, resulting in a 3 to 4 times less expensive test then presently available MRSA PCR kits. The less expensive SA PCR test is used to rule-out 70% of the samples, which are SA negative, resulting in an approximate overall 50% MRSA screening savings. These savings can be passed on to the consumer to enable a much more cost effective screening paradigm. With lower costs may follow broader implementation, resulting in a significant reduction in healthcare MRSA costs, as well as a reduction in morbidity.

Determination of SA negative samples in accordance with the present invention is assessed by direct PCR. Direct PCR in the general sample set is accomplished by an initial bacterial cell wall disruption. Surprisingly, it has been discovered that SA cell disruption and thus amplifiable DNA often exists naturally in nasal mucus samples and can be readily captured via nasal swabs. Equally surprising, it has been further discovered that by simply heating or freezing the nasal swab mucus sample, either by itself or in aqueous based buffers, further increases the proportion of disrupted SA cells and thus amplifiable DNA. Furthermore, the cells which are disrupted naturally by the nasal mucosal defense mechanisms and/or by freeze thaw and heating cycles, provide amplifiable SA DNA at diagnostically relevant levels compared to the gold standard of culture detection. SA cell disruption can be further accomplished by such techniques well known by those skilled in the art, such as through enzymatic cell wall lysis, achromopeptidase (ACP) proteinase K, Lysozyme, autolysin, sonication wave energy (sonication), electrolysis, pulsed electric field (PEF), electroporation, bead mill homogenizers, centrifugation, ionic or non-ionic detergents, combinations of each, or any means of successful SA cell disruption known in the art. Preferably, in the practice of the present invention, techniques such as inherent natural lysis, high temperature lysis, low temperature lysis, electroporation, sonication, bead mill, Saponin, quaternary alkyl amines such as NIMBUS and nisin antibiotic are all contemplated, as well as combinations thereof.

Further, in the practice of the present invention, elimination of PCR inhibitors can be accomplished by utilization of agents such as IgG(s), mucin(s), glycoproteins, nasal RX, blood, heat denaturation, activated charcoal, activated carbon, rapid hybridization, or by any other means known to those skilled in the art. The present invention also contemplates performing on a nasal sample certain procedures prior to cell wall disruption, followed by direct PCR, such as including, but not limited to, procedures well known in the art such as immunomagnetic enrichment with protein A antibodies, IgG bead binding to SA protein A, thermostable nuclease nuc antibodies, coagulase antibodies, fibronectin FN binding, fibronectin surface binding protein(s), or combinations thereof.

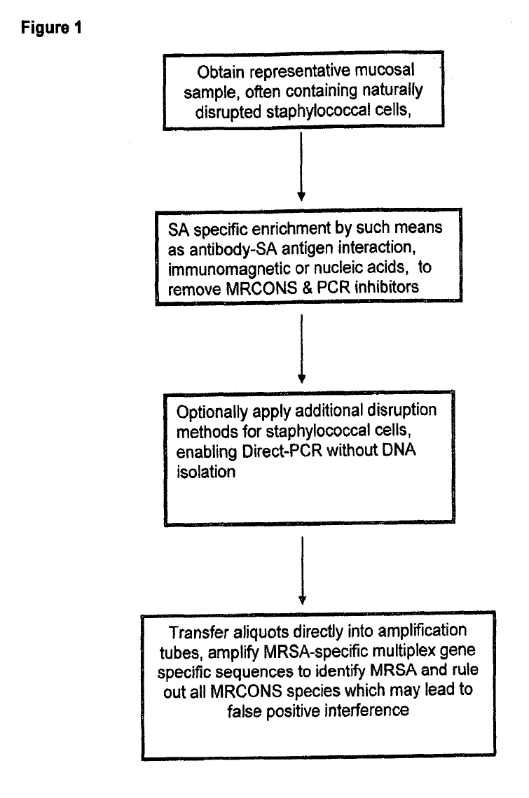

DNA extraction and isolation in the practice of the invention can be accomplished by means well known in the art, with the selection algorithm in FIG. 1 being particularly advantageous, instead of a direct PCR, in an especially preferred embodiment of the present invention.

Genes targeted in any of the amplification steps of the present invention include all of those known in the art for SA or MRSA identification. For example, femA, nuc, sa442, or tufA can be used as SA specific genes, and in SA immunomagnetic procedures, detection of mupirocin resistance uses ileS-2. Coagulase negative Staphylococcus (CONS) are endogenous to human nasal mucosa and are herein considered in connection with the invention as an inherent target for an overall process control in these methods, compositions and kits according to the invention, especially applying the tufA specific gene targets.

Amplification assays useful in the present invention include but are not limited to DNA amplification assays, PCR assays incorporating thermostable polymerases, and isothermal amplifications methods.

As mentioned previously, SA direct PCR, as comtemplated in the present invention, can be part of a more cost effective and rapid screening test compared to previously described tests of the conventional art, such as the aforedescribed FDA-approved MRSA PCR tests. Initially, it has been found that SA direct PCR will identify SA carriers to rule-out approximately 70% of the general sample population pool (MRSANRSA suspect population), resulting in approximately a 50% reduction in screening costs. This improved screening algorithm, as illustrated in FIG. 1, can result in significant cost savings and as such enables the adoption of broader screening and concomitantly fewer SA/MSSA/MRSANRSA associated deaths.

Thus, the present invention contemplates providing cost saving improvements over current, conventional PCR antibiotic resistant SA screening tests of the art, especially for MRSA and VRSA. These improvements involve, in part, the incorporation of “direct” nasal SA sample preparation methods applied in combination with a selection process for MRSA and/or VRSA. This selection process utilizes bacterial population demographics such as, but not limited to, the data suggesting that only about 30% of the human population at any one time has nasal colonization with SA. Direct nasal SA sample preparation involves the disruption and liberation of bacterial genomic DNA, specifically SA genomic DNA, but without DNA extraction. Instead of purifying DNA, a disrupted sample is directly transferred to a SA specific PCR reaction mix for testing. The direct sample prep results in a significant savings in total testing time before a result is obtained, reduction in operator hands-on time and a reduction in the reagents and equipment normally used to extract and isolate genomic DNA. The significant reduction in operator hands-on time not only achieves significant cost savings and time to results, but also significantly reduces overall assay complexity and thus contamination potential due to less open tube manipulations. All of the foregoing will be seen to those skilled in the art as advantageous results of the practice of the present invention, by comparison with conventional techniques of the art.

The present invention still further provides for the use of different SA antibody-antigen complexes forming combinations, to selectively enrich for SA and to selectively eliminate all of the known species, and as yet any unknown, MRCONS species.

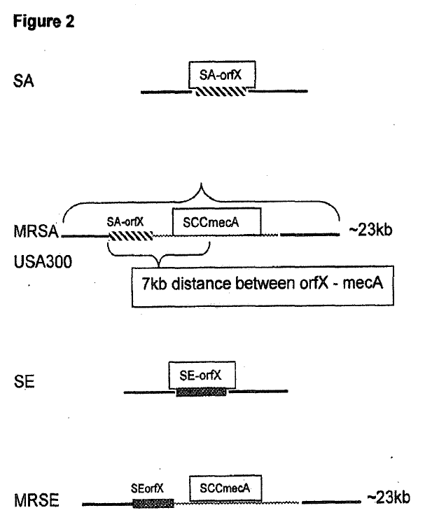

These alternatives can include, but are not limited to, IgG bead binding of protein A on all SA cells, thermostable nuclease nuc antibodies, coagulase antibodies, and fibronectin binding solid support systems in combination with fibronectin surface binding proteins found on SA. The present invention contemplates the same principles with the use of nucleic acid based enrichment of SA DNA from all MRCONS DNA, after nasal cell disruption and liberation of the genomic DNA, using disrupted cells and SA DNA specific hybridization methods. One such method can be defined as “sequence-specific enrichment of fragmented genomic DNA.” It is well known that all genomic DNA (gDNA) isolations and physical manipulations of lysed cells cause random shearing of prokaryotic and eukaryotic gDNA from millions of base pairs (bp) in length, down to narrow size range of 20,000 to 25,000 bp (20-25 kb). This fragment size (20-25 kb) can be considered an important physical linkage limitation unit, by which flanking sequences can be postulated to be “co-isolated”. MRSA is defined as SA strains that acquire the mobile genetic element SCCmecA which always inserts at a specific sequence adjacent to the orfX gene called attBscc. SCCmecA can vary in size and sequence composition designating at least six types, ranging in size from 28 kb-66 kb. In all of these types the mecA gene is within 10 kb of the orfX gene except for Type III where this distance is ˜40 kb. Fortunately Type III is a rare clinical isolate; however type III's mecA gene is located within 20 kb of the opposite side of orfX, and thus would be accessible via SA-specific probes from this opposite side. In addition the average fragment length could be increased above ˜23 lb by applying more gentle isolation techniques such as offered by ACP cell wall lysis, which is well known for preparing SA whole chromosomes for pulse field gel electrophoresis. For all types except III, the GenBank MRSA strain called USA300 a type IVa is illustrated here by way of example of the practice of the invention, but not limitation, as shown in FIG. 2 as a general model for orfX based capture from routinely manipulated gDNA fragments of ˜23 kb. The sequenced GenBank genome map of USA300 shows the physical linkage distance of the SA-orfX SCCmec insertion site and the mecA gene are only 7,000 bp (7 kb). Thus, on average the most gDNA fragments from USA300 captured via SA-orfX hybridization will be physically linked to the flanking mecA gene. It may be possible that a 7 kb PCR could be developed to demonstrate this linkage of SA-orfX to mecA defining MRSA, but a novel approach for demonstrating the presence of MRSA gDNA in accordance with the present invention is to first enrich for a SA-sequence specific sequence (e.g. using a SA-orfX specific capture probe) from a routine sample prepared containing SA-gDNA (20 kb minimum fragment size), where the majority of SA-orfX containing fragments will on average still be physically linked to SCCmecA gene if MRSA gDNA existed in the sample. After successful enrichment of SA-orfX captured fragments away from any similar sequences containing a confounding SCCmecA such as are routinely found in all CONS, and especially S. epidermidis in nasal samples, a simple quantitative tiplex analysis for a single copy SA-specific sequence (in this example orfX), a single copy SE-specific sequence (again SE-specific-orfX) and a third analyte a SSCmecA-specific sequence (in this example mecA), may prove the presence of MRSA. The MRSA presence criteria would, for example, be ratio quantities as follows:

1. If quant SA-orfX>=quant mecA>quant SE-orfX, then MRSA present.

2. If quant of mecA>=SA-orfX>SE-orfX, then MRSA

3. If quant of SE-orfX>=SA-orfX, then invalid enrichment failed.

Thus, a successful hybridization enrichment assay targeting a specific sequence fragment requiring enrichment from a confounding mixture of fragments would contain three critical elements. First, specific hybridization of appropriately fragmented and single strand denatured sample gDNA enabling desired linkage of flanking sequences; second, capture and enrichment of the specific target fragment; and third, a multiplex relative quantitative assay system to show the ratio of enriched target and sufficient elimination of confounding contamination targets, thereby enabling proof of the presence or absence of specific target sequence(s). Hybridization could be accomplished by any traditional means, as will be well known to those skilled in the art. In addition to current conventions of synthetically synthesized oligonucleotides hybridization probes there are contemplated for use in the present invention new probe types offering superior performance properties such as higher target affinity and less salt and temperature dependence. Examples of two of these are peptide nucleic acid (PNA) and locked nucleic acids (LNA), both of which could be substituted for DNA capture hybridization probes and/or used in combination to create duplex DNA capture structures called PD-Loop complexes.

Another novel technique contemplated by the present invention is the optimization of hybridization conditions, such that target fragment Tm is approached in such a manner as to enable only a local regional (or partial) melting of the probe area, while never achieving complete double stranded dissociation. The double-stranded fragment thus obtained is then maintained in a steady state equilibrium of partial denaturation, and annealing would now be receptive to oligonucleotides DNA capture probe hybridization, forming duplex D-loop complex for downstream enrichment. Likewise, PNA and LNA probes could be substituted, likely facilitating a greater extent of target duplex D-loop formation, capture and enrichment. One critical advantage of capturing double stranded duplex gDNA fragments is the avoidance of SA-SE heteroduplex formation capture complex, which would confound the subsequent assay analysis. It is further anticipated that in accordance with the present invention any of these capture probes can be modified with a capture moiety such as biotin and the like, enabling subsequent capture and enrichment via streptavidin coated beads, preferably magnetic beads. Following capture and washing to enrich and concentrate the target, specific assay(s) would be applied to prove enrichment and presence of the target, as previously described. One particularly preferred assay system is multiplex amplification via quantitative PCRm as previously described, however, it is to be appreciated that isothermal and other nucleic acid systems could also achieve the same result.

An additional modification of the three element system described above could be reduced to a two element system (hybridization+assay) by using the sequence-specific hybridization probes to deliver a sequence-specific cross-linking agent(s) only to the target(s) of interest. In this case, PNA and LNA will likely offer the most potential due to their improved performance properties relative to conventional DNA/RNA based probes. The first element of this system entails cross-linking the double stranded fragment of specific sequences, using compounds such as cisplatin, transplatin and psoralen linked to and delivered by hybridization probes. Cross-linking is conducted according to the well known and established DNA cross-linking chemistry conditions, such as UV light etc. After sequence specific cross-linking is complete, the complex mix of gDNA fragments is heated to boiling in aqueous solution to achieve complete complimentary strand denaturation, except for the specific cross-linked double stranded fragments. The entire mixture is then rapidly cooled under thermal conditions, such that only the specific cross-linked strands are re-annealed back to their original double stranded from as kinetically directed by the cross link. All other non-cross-linked strands are unable to find their original complimentary strand and thus remain in a predominantly single stranded state. To this mix, a single strand specific nuclease is then added to completely digest all DNA except the specific targeted fragment existing in its cross-linked, directed double stranded form. Then an aliquot is placed in an appropriate multiplex amplification system, such as the conventional systems described above, to identify the presence or absence of target.

The present invention further considers the use of immunomagnetic enrichment in nasal samples for the removal of known nasal derived PCR inhibitors such as mucus, IgG, blood, nasal treatments and drugs, all contributing to False Negative PCR results.

Accordingly, although the foregoing invention has been described in some detail by way of illustrations and examples for purposes of clarity of understanding, it will be readily apparent to those of ordinary skill in the art in light of the teachings of this invention that certain changes and modifications may be made thereto without departing from the spirit or scope thereof, and the following examples of procedures conducted in accordance with preferred embodiments of the invention are provided in the way of further illustration of the invention, but not limitation.

### Example 1

### Achromopeptidase Disruption of the SA Cell Wall, Compatible with Direct-PCR & Nasal Swab Samples Containing PCR Inhibitors

Nasal samples were obtained from nasal swabs after elution with 200 micro liters of TE. Samples were then incubated with or without achromopeptidase (ACP) incubation at 1 Unit/ul 37 C for 15 minutes followed by 99 C for 5 minutes. Direct TaqMan PCR amplification of an exogenous spiked in control template DNA at a volume of up to 2.5 micro liters of this ACP lysate in a 25 micro liter PCR reaction, confirmed compatibility. Further, transfer of volumes greater than 2.5 ul in to the 25 ul PCR showed inhibition from both sample types, suggesting that inhibition might start to negatively effect PCR above this volume proportion if not removed. Thus, it has been shown that in accordance with the present invention, ACP Direct PCR from nasal swab samples can be improved by removal of PCR inhibitors using methods such as cell, or DNA enrichment, activated charcoal etc. as described previously.

### Example 2

### ACP Followed by Qiagen Silica DNA Isolation

When the above-described ACP disruption system was performed on TE buffer spiked with varying CFU numbers of SA strain ATCC-29213, and then followed by Qiagen Micro kit DNA isolation, the reproducible lower limit measured by TaqMan nuc137 real-time PCR was less than or equal to 10 colony forming units (CFU). These sample amplification results are consistent with and suggest that the vast majority of SA cells are also disrupted due to ACP treatment.

### Example 3

### Prevalence of Nasal SA by Culture and PCR

In a preliminary study using routine SA culture methods, 15 random subjects were tested for nasal swab SA and 4 subjects were shown to be positive by Culture resulting in a prevalence of SA at 27%. This same n=15 sample set was also disrupted by ACP (1 u/ul), after being eluted in TE (10 mM, 1 mM EDTA), by vortexing the nasal swab for 1 minute. DNA was then isolated using the commercially available Qiagen Micro kit and SA specific TaqMan nuc137 real-time PCR showed 100% concordance with the culture results. Process blanks and controls indicated that no contamination was present during this study. This SA prevalence number is in agreement with the expected percentage found in the literature.

### Example 4

### Disrupted Nasal Swab Derived SA by Other Methodologies

Further disruption methods through boiling, freeze thawing and the possibility of an inherently amplifiable SA DNA were evaluated for use in the present invention, from nasal swab derived SA specimens in combination with Direct PCR. With the persistently positive and negative nasal SA carriage subjects as identified in Example 3 the above-established ACP disruption method was compared to three new disruption sample preparation methods for compatibility with Direct-PCR. Each of four subjects (two positive & two negative) was swabbed and then eluted by vortexing into TE yielding 300 ul of TE swab eluate. 50 ul of eluate was then disrupted for each the following four methods: ACP, boiling, freeze thawing and no treatment (or inherent to sample). 1.25 ul of each of these four treatments was then transferred to a 25 ul SA specific nuc137 TaqMan real-time PCR reaction and amplified for 45 cycles relative to standard curve, no template master mix controls and process blanks for the entire procedure excluding mucus sample. All contamination controls were found to be negative for nuc137. The two previous SA negative samples were again negative for all four treatments via nuc137. The two previous SA positive samples were found to be both positive by Direct-PCR for ALL four treatments, including the untreated “inherent” samples, thereby demonstrating that PCR amplifiable DNA are inherent to nasal mucosal SA and likely all flora.

### Example 5

### Immunomagnetic and DNA, PNA, LNA Probe Based SA Enrichment

Immunomagnetic enrichment in accordance with the present invention is contemplated prior to sample disruption, and Direct PCR can be expected to improve Direct PCR by eliminating potential PCR inhibitors. Thus, any protocol that enriches for the SA bacteria, live or dead, or the nucleic acids thereof, will in theory improve the analytical sensitivity and accuracy of the Direct PCR approach.

### Example 6

### Consequences of Identifying Persistently Positive/Negative Groups

It is to be appreciated that the majority of SA carriage positive and negative individuals are persistently so, at a constant rate of approximately 30% prevalence. It is believed that this persistent prevalence rate is due to some as yet uncharacterized human factor(s). Thus, once these persistent positive and negative groups are identified, the need to actively test the general population may be reduced to about the 30% persistent level plus a minor group of transitory individuals.

While certain of the preferred embodiments of the present invention have been described and specifically exemplified herein, it is not intended that the invention be limited to such embodiments. Various modifications may be made thereto as will be apparent to those skilled in the art, without departing from the spirit and scope of the present invention, and the full scope of the present invention is delineated in the following claims.

We claim:

1. A method for the detection of methicillin-resistant forms of Staphylococcus aureus (MRSA) from a sample population comprising:
a. obtaining a sample from a subject suspected of containing Staphylococcus aureus cells;
b. enriching said cells;
c. disrupting said cells so as to release their DNA;
d. detecting the presence of Staphylococcus aureus and Staphylococcus epidermidis in a multiplex PCR reaction; and
e. analyzing for the presence of MRSA by determining the non-Staphylococcus epidermidis, methicillin-resistant, coagulase negative staphylococci aureus cells.

2. The method of claim 1 wherein said sample is selected from a group consisting of a nasal swab, a nasopharyngeal swab, an inguinal swab, an anal swab, an ear swab, and combinations thereof.

3. The method of claim 1 wherein said enriching uses immunomagnetic enrichment from samples.

4. The method of claim 1 wherein said enriching uses nucleic acid hybridization capture probes composed of DNA, RNA, PNA, LNA to enrich for Staphylococcus aureus genomic DNA.

5. The method of claim 1 wherein known PCR inhibitors are removed from said cells prior to enrichment.

6. The method of claim 5 wherein said detecting of Staphylococcus aureus in said enriched samples is by amplification of mecA gene and femA gene.

7. The method of claim 5 wherein said detecting of Staphylococcus aureus in said enriched samples is by amplification of mecA gene and nuc gene.

8. The method of claim 1 wherein Staphylococcus aureus and Staphylococcus epidermidis and CONS are detected in a multiplex PCR reaction by quantifying mecA gene, femA gene, and tufA gene.

9. A kit for the detection of methicillin-resistant forms of Staphylococcus aureus (MRSA) from a sample population comprising:
a. a sample from a subject suspected of containing Staphylococcus aureus cells;
b. means for removing known PCR inhibitors from said sample;
c. means for enriching said cells and or genomic DNA wherein said cells are devoid of PCR inhibitors;
d. means for performing a multiplex PCR reaction to detect Staphylococcus aureus and Staphylococcus epidermidis; and
e. means for analyzing the presence of MRSA by determining the non-Staphylococcus epidermidis, methicillin-resistant, coagulase negative staphylococci aureus cells

10. The kit in claim 7 wherein Staphylococcus aureus and Staphylococcus epidermidis are detected in a multiplex PCR reaction by quantifying mecA gene, femA gene, nuc gene, and tufA gene.
